(19) United States
(12) Patent Application Publication
(10) Pub. No.: US 2026/0294883 A1
(43) Pub. Date: Oct. 1, 2026

(54) PHARMACEUTICAL COMPOSITION FOR THE TREATMENT OF POST-TRAUMATIC STRESS SYNDROME

(71) Applicant: KINNOV THERAPEUTICS, Orleans (FR)

(72) Inventors: Fabrice TROVERO, Avaray (FR); Philippe BERNARD, La Ferté Saint Aubin (FR); Jean-Pol TASSIN, Clamart (FR)

(73) Assignee: KINNOV THERAPEUTICS, Orleans (FR)

(21) Appl. No.: 18/998,191

(22) PCT Filed: Jul. 26, 2023
(86) PCT No.: PCT/FR2023/051161

§ 371 Date: Jan. 24, 2025

(30) Foreign Application Priority Data
Jul. 26, 2022 (FR) FR2207642

(51) Int. Cl. A61K 31/4418; A61K 31/18; A61K 31/404; A61K 31/517; A61P 25/00

(52) U.S. Cl. CPC A61K 31/4418; A61K 31/18; A61K 31/404; A61K 31/517; A61P 25/00

(57) ABSTRACT

A pharmaceutical composition in the treatment of the post-traumatic stress syndrome in humans includes: a compound having an antagonist action on serotonergic 5-HT2 receptors selected as cyproheptadine; and a compound having an antagonistic action on alpha1-noradrenergic receptors chosen among prazosin, alfuzosin, terazosin, tamsulosin, silodosin and doxazosin.

The present invention relates to a pharmaceutical composition for the treatment of post-traumatic stress syndrome in humans.

Post-traumatic stress syndrome (PTSS), also known as post-traumatic stress disorder (PTSD), post-traumatic stress state (PTSS), denotes a severe anxiety disorder that generally manifests itself following an experience perceived as traumatic with a confrontation with ideas of death. It is a bio-neuro-physiological reaction following a situation during which the physical or psychological integrity of the patient, or that of his entourage, has been threatened or actually affected.

It can have very diverse origins such as war, a serious accident, a violent death, a serious illness, or sometimes even a trauma at the time of birth.

Patients with post-traumatic stress syndrome typically complain of a feeling of hopelessness or horror associated with a triad of persistent symptoms, including:
intrusion—the patient relives the traumatic event and feels the anxiety felt at the time of the event;
avoidance: the patient does everything to avoid situations and triggers that could remind him of the traumatic event
hyperstimulation: the patient suffers from several symptoms of hypervigilance which include difficulty concentrating, insomnia, nervousness, a constant feeling of danger or imminent disaster and great irritability or even violent behavior.

According to some studies, more than 70% of adults in the world experience or have experienced a traumatic event during their life, and a certain number of them (which can reach up to 12% of the population in certain countries in difficulty) suffer from post-traumatic stress.

At the date of the present invention, there is no standard and recognized treatment for post-traumatic stress syndrome in humans. The treatment, particularly pharmacological, of this syndrome has therefore been an important issue in public health for many years.

At the date of the present invention, different types of treatments, whether medicinal or not, are implemented with a view to treating post-traumatic stress syndrome.

Non-medicinal treatments include psychotherapy treatments that are offered as first-line treatment, but also cognitive-behavioral therapies, hypnosis, transcendental meditation, and play therapy. These treatments generally target anxiety and depressive disorders.

When these treatments have proven ineffective, for example in cases of chronic, long-term disorders with associated comorbidity (depression being very often present in chronic post-traumatic stress disorders), medicinal treatments may be indicated.

Various studies have been conducted to identify effective medicinal treatments for post-traumatic stress syndrome. Some of the treatments that have been the subject of such studies and are most commonly recommended include:
the use of SSRI-type antidepressants such as citalopram, escitalopram, fluoxetine, fluvoxamine, paroxetine, and sertraline;
the use of SNRI-type antidepressants such as venlafaxine
the use of anxiolytics for anxiety and sleep disorders, such as zolpidem.
the use of beta-blockers, such as propranolol, or alpha-blockers such as prazosin;
the use of alpha-2 norepinephrine receptor agonists, such as clonidine;
the use of antihistamines such as hydroxyzine and cyproheptadine; and
the use of certain antipsychotics such as quetiapine.

However, at the date of the present invention, only two antidepressants are officially indicated by the authorities in the treatment of post-traumatic stress: these are paroxetine (notably marketed under the name Deroxat® or Divarius®) and sertraline (notably marketed under the name Zoloft®). Unfortunately, their effectiveness remains limited since only 20 to 50% of patients show a reduction in symptoms. On the other hand, antidepressants have side effects such as sleep disorders, headaches, sexual disorders, weight gain, dizziness, etc. which are very poorly accepted in the context of post-traumatic stress syndrome pathology.

In addition, the symptomatological complexity of post-traumatic stress syndrome and the associated comorbidity make the use, often in combination, of these different substances critical or even dangerous. For example, the combination of anxiolytics and antidepressants increases the risk of disinhibition that can lead to suicide or misuse in patients with high risk of consuming addictive substances such as alcohol or drugs.

At the date of the present invention, it therefore remains necessary to develop new medicinal treatments to effectively combat post-traumatic stress syndrome while limiting the poly-consumption of psychotropic substances.

Cyproheptadine denotes 4-(5-H-dibenzo[a,d]cyclohepten-5-ylidene)-1-methylpiperidine hydrochloride of the formula:

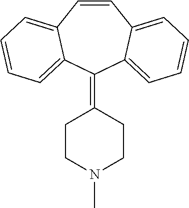 [SMILES: CN1CCC(=C2c3ccccc3C=Cc3ccccc32)CC1]

This active ingredient is known to have an antagonistic action on serotonergic 5-HT2 receptors and is used usually as an antihistamine/anticholinergic and antiserotonergic. Marketed under the name Periactine®, it is recommended for the symptomatic treatment of various allergic manifestations.

Prazosin denotes 2-[4-(2-furoyl)piperazin-1-yl]-6,7-dimethoxyquinazolin-4-amine of the formula:

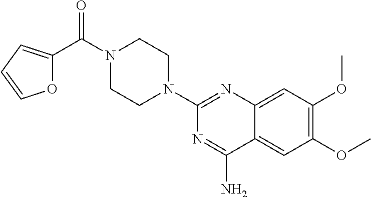 [SMILES: COc1cc2nc(N3CCN(C(=O)c4ccco4)CC3)nc(N)c2cc1OC]

This active ingredient is known to have an antagonistic action on alpha1-noradrenergic receptors and is usually used as a hypotensive agent. Marketed under the name Minipress®, it is recommended for treating high blood pressure and congestive left ventricular failure, but also as part of the symptomatic treatment of Raynaud's phenomena (primary or secondary) and certain functional manifestations related to benign prostatic hypertrophy.

Alfuzosin denotes N-[3-[(4-amino-6,7-dimethoxy-quinazolin-2-yl)-methyl-amino]propyl]tetrahydrofuran-2-carboxamide of the formula:

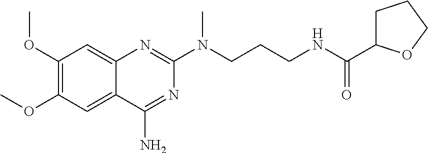 [SMILES: COc1cc2nc(N(C)CCCNC(=O)C3CCCO3)nc(N)c2cc1OC]

This active ingredient is known to have an antagonistic action on alpha1-noradrenergic receptors. Marketed under the name Urion®, it is recommended to treat the functional symptoms of benign prostatic hypertrophy.

Terazosin denotes 6,7-dimethoxy-2-[4-(tetrahydrofuran-2-ylcarbonyl)piperazin-1-yl]quinazolin-4-amine of the formula:

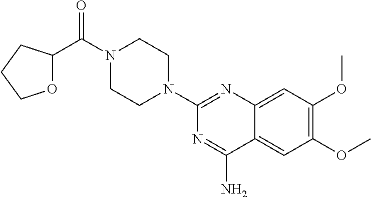 [SMILES: COc1cc2nc(N3CCN(C(=O)C4CCCO4)CC3)nc(N)c2cc1OC]

This active ingredient is known to have an antagonistic action on alpha1-noradrenergic receptors. Marketed under the name Hytrine® or Dysalfa®, it is recommended to treat the functional symptoms of benign prostatic hypertrophy.

Tamsulosin denotes (R)-5-(2-(2-(2-ethoxyphenoxy)ethylamino)propyl)-2-methoxybenzenesulfonamide of the formula:

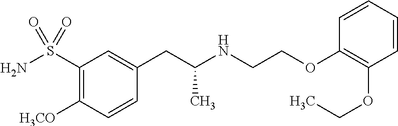 [SMILES: CCOc1ccccc1OCCN[C@H](C)Cc1ccc(OC)c(S(N)(=O)=O)c1]

This active ingredient is known to have an antagonistic action on alpha1-noradrenergic receptors. Marketed under the name Josir® or Emix®, it is recommended to treat the functional symptoms of benign prostatic hypertrophy.

Silodosin denotes 1-(3-hydroxypropyl)-5-[(2R)-({2-[2-[2-(2,2,2-trifluoroethoxy) phenoxy]ethyl}amino)propyl]indoline-7-carboxamide of the formula:

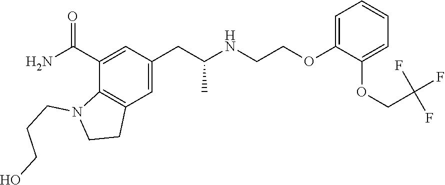 [SMILES: C[C@H](Cc1cc2c(c(C(N)=O)c1)N(CCCO)CC2)NCCOc1ccccc1OCC(F)(F)F]

This active ingredient is known to have an antagonistic action on alpha1-noradrenergic receptors; particularly the alpha1a subtypes; which may avoid potential problems of orthostatic hypotension. Marketed under the name Urorec® or Sylodix®, it is recommended to treat the functional symptoms of benign prostatic hyperplasia.

Doxazosin denotes (RS)-2-{4-[(2,3-dihydro-1,4-benzodioxin-2-yl)carbonyl]piperazin-1-yl}-6,7-dimethoxyquinazolin-4-amine of the formula:

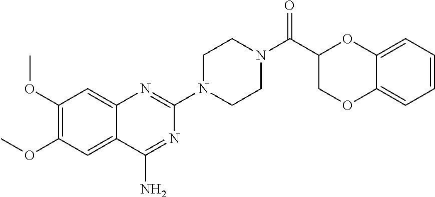 [SMILES: COc1cc2nc(N3CCN(C(=O)C4COc5ccccc5O4)CC3)nc(N)c2cc1OC]

This active ingredient is known to have an antagonistic action on alpha1-noradrenergic receptors. Marketed under the name Doxazosine TEVA LP®, it is recommended to treat the functional symptoms of benign prostatic hypertrophy and high blood pressure.

International patent applications WO-A-2011/080488 and WO-A-2012/175894 describe a pharmaceutical composition comprising a compound having an antagonistic action on serotonergic 5-HT2 receptors chosen as cyproheptadine and a compound having an antagonistic action on alpha1-noradrenergic receptors chosen from prazosin, alfuzosin, terazosin, tamsulosin, silodosin and doxazosin, for the treatment of dependence (in particular alcohol dependence) in humans. However, these patent applications make no mention of other therapeutic uses of these compositions, in particular for the treatment of post-traumatic stress syndrome.

In “The serotonergic and alpha-1 adrenergic receptor modulator ACH-000029 ameliorates anxiety-like behavior in a post-traumatic stress disorder model”, Neuropharmacology, 164 (2020) 107912, authors Azevedo et al. report the results of a study to compare the efficacy of ACH-000029, a compound with simultaneous antagonist action on alpha1-noradrenergic and serotonergic receptors (5HT1A, 5HT1D, 5HT2A), and that of prazosin in the treatment of post-traumatic stress syndrome. Based on the obtained results, the authors conclude that ACH-000029 is more effective than prazosin alone for the treatment of post-traumatic stress syndrome and suggest that the dual action on 5-HT and alpha1-noradrenergic receptors is necessary to alleviate the symptoms of post-traumatic stress syndrome. The authors indicate that this result is in line with the current use of antidepressants, these molecules often having this type of dual action on these neurotransmission systems. However, the reduced clinical efficacy of these treatments indicates that this action is not sufficient, and would only concern the anxiety disorders aspect of the pathology.

Indeed, to treat post-traumatic stress syndrome, it is not only necessary to consider the observed anxiety and depressive disorders, but also the phenomenon of memorization of the stressful event. The lifting of behavioral inhibition linked to the taking of antidepressant or anxiolytic molecules represents an aggravating factor in the risk of suicide. The translational relevance of preclinical models required by regulatory agencies for the characterization of molecules specifically active in post-traumatic stress syndrome therefore implies the use of different models including those taking into account the memorization aspect of the stressful event. Furthermore, the translational aspect of these models must also take into account the fact that, in most cases, the stressful event is unique and acute.

Thus, as indicated by the EMA in “Guideline on the Development of Medicinal Products for the Treatment of Post-Traumatic Stress Disorder (PTSD)”, CHMP/EWP/358650/2006 Corr 2*, July 2008, the demonstration of anxiolytic activity alone is not sufficient to validate a therapeutic action against post-traumatic stress syndrome.

Furthermore, cyproheptadine is never mentioned by the authors Azevedo et al.

It has now been found, quite surprisingly, that, contrary to what is observed with other compounds having an antagonistic action on serotonergic 5-HT2 receptors, there is a synergy of activity between cyproheptadine and certain compounds having an antagonistic action on alpha1-noradrenergic receptors in the treatment of post-traumatic stress syndrome compared to the activity of each of the compounds used alone. The combination of these active ingredients thus makes it possible to obtain rapid therapeutic efficacy that is superior to reference medicines. In addition, this efficacy is long-lasting since it continues after treatment is stopped.

Thus, the present invention relates to a pharmaceutical composition for use in the treatment of post-traumatic stress syndrome in humans comprising:
a compound having an antagonist action on serotonergic 5-HT2 receptors chosen as being cyproheptadine; and
a compound having an antagonistic action on alpha1-noradrenergic receptors chosen among prazosin, alfuzosin, terazosin, tamsulosin, silodosin and doxazosin.

The pharmaceutical composition according to the invention makes it possible to obtain a rapid therapeutic effect, superior to reference medicines, and lasting over time, including after stopping treatment.

In the context of the present invention:
“Post-traumatic stress syndrome” means any pathology characterized by the development of symptoms such as depression, generalized anxiety, inability to work, withdrawal from social life or sleep disorders in people exposed to major traumatic civilian events (accidents, assaults, rapes, attacks) or military events (combat, interventions);
“continuous schedule” means the continuous therapeutic treatment of a patient, comprising the successive administration of one or several therapeutic compositions (including multitherapies, according to the invention or not), identical or different, each with its own therapeutic administration scheme (number of daily administrations and number of days of administration over a given period, week for example) and this, in an unlimited manner and not sequenced or spaced out in time, that is to say without interruption of treatment;
“metabolite” of an active ingredient means any intermediate product resulting from the transformation of said active ingredient in the body during a metabolic process;
cyproheptadine denotes (5H-dibenzo[a,d]cyclohepten-5-ylidene)-1-methylpiperidine hydrochloride, as well as its pharmaceutically acceptable salts or derivatives;
“prodrug” of an active ingredient means any compound whose biotransformation in the body results in said active ingredient;
“pharmaceutically acceptable salt” of an active ingredient means any addition salt of said active ingredient with a mineral or organic acid by the action of such an acid in an organic or aqueous solvent such as an alcohol, a ketone, an ether or a chlorinated solvent, and which is acceptable from a pharmaceutical point of view;
“pharmaceutically acceptable derivative” of an active ingredient means any “prodrug” or “metabolite” of said active ingredient, as well as their pharmaceutically acceptable salt;
alfuzosin denotes N-[3-[(4-amino-6,7-dimethoxy-quinazolin-2-yl)-methyl-amino]propyl]tetrahydrofuran-2-carboxamide, as well as its pharmaceutically acceptable salts or derivatives;
prazosin denotes 2-[4-(2-furoyl)piperazin-1-yl]-6,7-dimethoxyquinazolin-4-amine, as well as its pharmaceutically acceptable salts or derivatives;
tamsulosin denotes (R)-5-(2-(2-(2-ethoxyphenoxy)ethylamino)propyl)-2-methoxybenzenesulfonamide, as well as its pharmaceutically acceptable salts or derivatives;

terazosin denotes 6,7-dimethoxy-2-[4-(tetrahydrofuran-2-ylcarbonyl)piperazin-1-yl]quinazolin-4-amine, as well as its pharmaceutically acceptable salts or derivatives;

silodosin denotes 1-(3-hydroxypropyl)-5-[(2R)-({2-[2-[2-(2,2,2-trifluoroethoxy) phenoxy]ethyl}amino)propyl]indoline-7-carboxamide, as well as its pharmaceutically acceptable salts or derivatives; and
doxazosin denotes (RS)-2-{4-[(2,3-dihydro-1,4-benzodioxin-2-yl)carbonyl]piperazin-1-yl}-6,7-dimethoxyquinazolin-4-amine, as well as its pharmaceutically acceptable salts or derivatives.

The pharmaceutical composition according to the present invention contains the active ingredients in sufficient quantity to ensure the desired therapeutic effect, that is to say the treatment of post-traumatic stress syndrome in the treated patient.

Preferably, the following daily amounts of active ingredients are used to prepare the pharmaceutical composition according to the invention:
from 0.04 to 50 mg of cyproheptadine, preferably from 0.4 to 20 mg of cyproheptadine;
from 0.025 to 40 mg of prazosin, preferably from 0.25 to 20 mg of prazosin;
from 0.075 to 20 mg of alfuzosin, preferably from 0.75 to 10 mg of alfuzosin;
from 0.01 to 10 mg of terazosin, preferably from 0.1 to 5 mg of terazosin;
from 0.004 to 1 mg of tamsulosin, preferably from 0.04 to 0.5 mg of tamsulosin;
from 0.05 to 50 mg of silodosin, preferably from 0.5 to 30 mg of silodosin;
from 0.025 to 20 mg of doxazosin, preferably from 0.25 to 10 mg of doxazosin.

Preferably, the pharmaceutical composition according to the present invention contains only two active ingredients.

Preferably, the subject of the present invention is a pharmaceutical composition as defined above, in which the compound having an antagonistic action on alpha1-noradrenergic receptors is chosen from prazosin, alfuzosin, terazosin and tamsulosin. Most preferably, the subject of the present invention is a pharmaceutical composition as defined above, in which the compound having an antagonistic action on alpha1-noradrenergic receptors is prazosin.

The pharmaceutical composition according to the present invention may be formulated in any galenic form necessary for its administration. In particular, with regard to oral administration, the compositions according to the present invention may be formulated in the form of coated or uncoated, effervescent, soluble, orodispersible, gastro-resistant or modified-release tablets; sugar-coated tablets; hard-shell capsules (or gelatin capsules); soft-shell capsules; granules; granulates; pills; lozenges. With regard to nasal administration, the composition may be formulated in the form of a spray or powder for inhalation. With regard to systemic administration, the composition according to the invention may be formulated in the form of a sterile lyophilized powder for injection. The pharmaceutical compositions according to the present invention may therefore comprise, in addition to the active ingredients, any pharmaceutically acceptable formulation adjuvant, known to those skilled in the art and which is necessary for the preparation of the pharmaceutical composition in the desired form.

The pharmaceutical composition according to the invention can be administered from one to four times per day, without exceeding the maximum daily dose. Thus, the present invention also relates to a pharmaceutical composition as defined above for administration 1 to 4 times per day to the patient suffering from post-traumatic stress syndrome.

The pharmaceutical composition according to the invention can be administered at any time of the day, before, during or after meals, without this affecting the effectiveness of the treatment.

The pharmaceutical composition according to the present invention can be administered to the patient one or more times per week. Thus, the present invention also relates to a pharmaceutical composition as defined above for daily administration 1 to 7 days per week to the patient suffering from post-traumatic stress syndrome.

The composition according to the present invention can be administered on a continuous schedule.

Another subject of the present invention also concerns the use of a pharmaceutical composition as defined above for the preparation of a medicament intended for the treatment of post-traumatic stress syndrome in humans.

Another subject of the present invention also concerns the use of a pharmaceutical composition as defined above for the preparation of a medicament intended for the treatment of post-traumatic stress syndrome in humans, said medicament being administered 1 to 4 times per day.

Another subject of the present invention also concerns the use of a pharmaceutical composition as defined above for the preparation of a medicament intended for the treatment of post-traumatic stress syndrome in humans, said medicament being administered daily 1 to 7 days per week.

The present invention also relates to a method for treating post-traumatic stress syndrome in a human being by administering a pharmaceutical composition as defined above.

The present invention also relates to a method for treating post-traumatic stress syndrome in a human being by administering 1 to 4 times per day a pharmaceutical composition as defined above.

The present invention also relates to a method for treating post-traumatic stress syndrome in a human being by daily administration 1 to 7 days per week a pharmaceutical composition as defined above, the administration possibly or not being carried out according to a continuous schedule.

The two active ingredients constituting the new pharmaceutical composition according to the invention can be administered in the form of a unit pharmaceutical composition comprising the two active ingredients allowing an administration of said composition to the patient in a single dose.

However, a separate administration of the two active ingredients constituting the new pharmaceutical composition according to the invention can also be envisaged.

Thus, the present invention also relates to a pharmaceutical product containing:
a compound having an antagonist action on serotonergic 5-HT2 receptors chosen as being cyproheptadine; and
a compound having an antagonist action on alpha1-noradrenergic receptors chosen among prazosin, alfuzosin, terazosin, tamsulosin, silodosin and doxazosin;
as a combination product (or pharmaceutical kit) for a simultaneous, separate or phased administration in the treatment of post-traumatic stress syndrome in humans.

The pharmaceutical product according to the invention can of course be administered according to one of the administration schemes defined above. Thus, the present invention also relates to a pharmaceutical product containing:
a compound having an antagonist action on serotonergic 5-HT2 receptors chosen as being cyproheptadine; and
a compound having an antagonist action on alpha1-noradrenergic receptors chosen among prazosin, alfuzosin, terazosin, tamsulosin, silodosin and doxazosin;
as a combination product (or pharmaceutical kit) for a simultaneous, separate or staggered administration 1 to 4 times per day, in the treatment of post-traumatic stress syndrome in humans.

The pharmaceutical product according to the invention can of course be administered according to one of the administration schemes defined above. Thus, the present invention also relates to a pharmaceutical product containing:
a compound having an antagonist action on serotonergic 5-HT2 receptors chosen as being cyproheptadine; and
a compound having an antagonist action on alpha1-noradrenergic receptors chosen among prazosin, alfuzosin, terazosin, tamsulosin, silodosin and doxazosin;
as a combination product (or pharmaceutical kit) for a simultaneous, separate or spread daily administration over time 1 to 7 days per week for the treatment of post-traumatic stress syndrome in humans.

The present invention is illustrated in a non-limiting manner by the following examples.

### EXAMPLE 1—COMPOSITION ACCORDING TO THE PRESENT INVENTION

### Experimental Protocol

The conditioned fear test in rodents is recognized as a relevant preclinical model for the study of post-traumatic stress disorders.

During this test, animals are placed for a 4.5-minute conditioning session in a square box with a stainless steel mesh floor connected to a shock generator.

A conditioned stimulus (CS) consisting of a sound (80 dB, 2800 Hz, 30 seconds) is presented twice (at 1.5 min and then at 3.5 min).

An unconditioned stimulus consisting of an electric shock to the paws is delivered at the end of each CS (2 seconds, 0.6 mA).

The animals were then subjected to memory test sessions of the stressful event on different days after the session of packaging. The sessions of test of memorization (duration of 6 min) comprise a first period of 3 minutes without none CS presentation (period CS−) followed by a second 3 minute period during which the CS is presented (period CS+).

The sound stimulus alone triggers conditioned fear behavior. In rodents, this behavior is manifested by a prostrate attitude, an immobility behavior (i.e. «freezing») that is distinguished from the animal's exploratory behavior when placed in an enclosure for a short period.

The analyzed data are the time of immobility during the period CS− and during the period CS+. The freezing index is defined as the difference in immobility time between the period with the sound stimulus and the period without the sound. Therefore, the higher this index, the greater the post-traumatic stress state.

The «sham» group received no shock. The treatments were administered immediately after the conditioning session, and then 30 minutes before each conditioned fear session (no shock, but with the conditioned stimulus).

This test measures the effect of stress and its environment and its memorization (i.e. extinction).

### Results

The results obtained with measurements taken 3 days after the conditioning session are reported in the following Table 1.

TABLE 1

| Groups | Freezing index | | |
|---|---|---|---|
| (n = 10 animals | (in seconds - | % vs | |
| per group) | Mean ± SEM) | witness | p |
| Sham | −2.03 ± 4.86 *** | — | p ≤ 0.001 |
| Witnesses | 62.12 ± 9.93 | 100.00 | — |
| Prazosin (0.5 mg/kg) | 41.97 ± 5.82 | 67.57 | ns |
| Prazosin (1 mg/kg) | 35.29 ± 12.22 | 56.80 | ns |
| Cyproheptadine (0.5 | 38.68 ± 5.84 | 62.28 | ns |
| mg/kg) | | | |
| Prazosin (0.5 mg/kg) + | 9.21 ± 6.16 ** | 14.83 | p ≤ 0.01 |
| Cyproheptadine (0.5 | | | |
| mg/kg) | | | |

p-value, versus control group
*** p ≤ 0.01;
** p ≤ 0 01;
* p ≤ 0.05
ESM: Standard Error of the Mean

### Conclusion

The obtained results show that prazosin, when administered alone, does not allow the treatment of post-traumatic stress syndrome.

The same is true when cyproheptadine is administered alone.

On the other hand, when these products are administered in combination (prazosin combined with cyproheptadine), a very clear reduction in the syndrome is observed.

There is therefore a synergy of activity between prazosin and cyproheptadine in the treatment of post-traumatic stress syndrome.

### EXAMPLE 2—COMPARISON BETWEEN A COMPOSITION ACCORDING TO THE PRESENT INVENTION AND COMPOSITIONS CONTAINING ANOTHER COMPOUND HAVING AN ANTAGONIST ACTION ON SEROTONERGIC 5-HT2 RECEPTORS

### Experimental Protocol

The protocol is identical to that of Example 1 above. In this example, the conditioned fear sessions and the «freezing» measurement are carried out on days 1, 3 and 4 after the conditioning session. The treatment is stopped after day 3, so that during the 4th observation session, no treatment is administered.

### Results

The obtained results are reported in the following Table 2.

TABLE 2

| Groups | Freezing index (in | | |
|---|---|---|---|
| (n = 10 animals | seconds - Mean ± SEM) | | |
| per group) | Day 1 | Day 3 | Day 4 |
| Witnesses | 39.0 ± | 40.2 ± | 35.6 ± |
| | 9.4 | 8.5 | 7.6 |
| Prazosin (0.5 mg/kg) + mianserin | 17.6 ± | 17.1 ± | 20.6 ± |
| (2 mg/kg) | 8.2 | 9.7 | 6.1 |
| Prazosin (0.5 mg/kg) + mianserin | 49.5 ± | 29.9 ± | 26.9 ± |
| (20 mg/kg) | 10.7 | 10.2 | 10.8 |
| Prazosin (0.5 mg/kg) + eplivanserin | 25.1 ± | 31.9 ± | 30.6 ± |
| (0.5 mg/kg) | 4.2 | 4.3 | 5.6 |
| Prazosin (0.5 mg/kg) + eplivanserin | 17.3 ± | 35.7 ± | 36.6 ± |
| (1 mg/kg) | 4.8 | 5.3 | 4.3 |
| Prazosin (0.5 mg/kg) + cyproheptadine | 5.5 ± | 8.3 ± | −2.5 ± |
| (0.5 mg/kg) | 6.0** | 6.0** | 7.5** |

p-value, versus control group
*** p ≤ 0.01;
**p ≤ 0 01;
* p ≤ 0.05
ESM: Standard Error of the Mean

### Conclusion

The obtained results show that, while there is a synergy of activity between prazosin and cyproheptadine in the treatment of post-traumatic stress syndrome, no synergy is observed between prazosin and other antagonists on serotonergic 5-HT2 receptors such as mianserin and eplivanserin.

### EXAMPLE 3—COMPARISON BETWEEN A COMPOSITION ACCORDING TO THE PRESENT INVENTION AND A REFERENCE COMPOSITION CONTAINING PAROXETINE

The protocol is identical to that of Example 1 above. In this example, the conditioned fear sessions and the freezing measurement are carried out on days 1, 3 and 4 after the conditioning session. The treatment is stopped after day 3, so that during the 4th observation session, no treatment is administered.

### Results

The obtained results are reported in the following Table 3.

TABLE 3

| | |
|---|---|
| Groups | Freezing index (in |
| (n = 10 animals | seconds - Mean ± SEM) |

| per group) | Day 1 | Day 3 | Day 4 |
|---|---|---|---|
| Sham | 7.6 ± | −1.9 ± | 5.3 ± |
| | 2.6*** | 3.3** | 6.0** |
| Witnesses | 53.3 ± | 38.1 ± | 56.2 ± |
| | 5.7 | 8.7 | 9.8 |
| Paroxetine (4 mg/kg) | 64.5 ± | 44.3 ± | 38.9 ± |
| | 7.4 | 10.5 | 4.8 |
| Paroxetine (8 mg/kg) | 51.3 ± | 33.1 ± | 50.3 ± |
| | 9.4 | 12.7 | 12.1 |
| Prazosin (0.5 mg/kg) + | 19.8 ± | 11.0 ± | 5.3 ± |
| cyproheptadine (0.5 mg/kg) | 7.4*** | 6.5* | 7.9** |

p-value, versus control group
***p ≤ 0.01;
**p ≤ 0 01;
*p ≤ 0.05
ESM: Standard Error of the Mean

### Conclusion

The obtained results show that paroxetine has no significant effect on the memory component and impact of the stressful event, in terms of anxiety and fear.

Conversely, it is observed a significant activity of the cyproheptadine/prazosin combination, that is to say a significant reduction in the anxious reaction during the procedure of recalling the stressful situation.

This property indicates a specific therapeutic potential for this combination in the treatment of post-traumatic stress syndrome.

1. A pharmaceutical composition for use in the treatment of the post-traumatic stress syndrome in humans comprising:
a compound having an antagonist action on serotonergic 5-HT2 receptors chosen as being cyproheptadine; and
a compound having an antagonistic action on alpha1-noradrenergic receptors chosen among prazosin, alfuzosin, terazosin, tamsulosin, silodosin and doxazosin.

2. The pharmaceutical composition for use in the treatment of the post-traumatic stress syndrome in humans according to claim 1, wherein it comprises:
from 0.04 to 50 mg of cyproheptadine; and
from 0.025 to 40 mg of prazosin, from 0.075 to 20 mg of alfuzosin, from 0.01 to 10 mg of terazosin, from 0.004 to 1 mg of tamsulosin, from 0.05 to 50 mg of silodosin, or from 0.025 to 20 mg of doxazosin.

3. The pharmaceutical composition for use in the treatment of the post-traumatic stress syndrome in humans according to claim 1, wherein the compound having an antagonist action on alpha1-noradrenergic receptors is chosen among prazosin, alfuzosin, terazosin and tamsulosin.

4. The pharmaceutical composition for use in the treatment of the post-traumatic stress syndrome in humans according to claim 3, wherein the compound having an antagonist action on alpha1-noradrenergic receptors is prazosin.

5. The pharmaceutical composition for use in the treatment of the post-traumatic stress syndrome in humans according to claim 1 for administration 1 to 4 times per day to the patient suffering from the post-traumatic stress syndrome.

6. The pharmaceutical composition for use in the treatment of post-traumatic stress syndrome in humans according to claim 1 for daily administration 1 to 7 days per week to the patient suffering from the post-traumatic stress syndrome.

7. A pharmaceutical product for use in the treatment of the post-traumatic stress syndrome in humans containing:
a compound having an antagonist action on serotonergic 5-HT2 receptors chosen as being cyproheptadine; and
a compound having an antagonist action on alpha1-noradrenergic receptors chosen among prazosin, alfuzosin, terazosin, tamsulosin, silodosin and doxazosin; as a combination product for simultaneous, separate or spread out administration.
